(19) United States
(12) Patent Application Publication
(10) Pub. No.: US 2022/0319706 A1
(43) Pub. Date: Oct. 6, 2022

(54) A DRGS AUTOMATIC GROUPING METHOD BASED ON A CONVOLUTIONAL NEURAL NETWORK

(71) Applicant: ZHEJIANG UNIVERSITY, HANGZHOU, Zhejiang Province (CN)

(72) Inventors: JIAN WU, HANGZHOU, ZHEJIANG PROVINCE (CN); JINTAI CHEN, HANGZHOU, ZHEJIANG PROVINCE (CN); TINGTING CHEN, HANGZHOU, ZHEJIANG PROVINCE (CN); HAOCHAO YING, HANGZHOU, ZHEJIANG PROVINCE (CN); BIWEN LEI, HANGZHOU, ZHEJIANG PROVINCE (CN); XUECHEN LIU, HANGZHOU, ZHEJIANG PROVINCE (CN); QINGYU SONG, HANGZHOU, ZHEJIANG PROVINCE (CN); JIUCHENG ZHANG, HANGZHOU, ZHEJIANG PROVINCE (CN); XIAOHONG JIANG, HANGZHOU, ZHEJIANG PROVINCE (CN)

(21) Appl. No.: 17/627,622

(22) PCT Filed: Nov. 12, 2020
(86) PCT No.: PCT/CN2020/128369

§ 371 Date: Jan. 14, 2022

(30) Foreign Application Priority Data
Dec. 18, 2019 (CN) 201911310269.9

(51) Int. Cl. G16H 50/20; G16H 50/70; G16H 40/20

(52) U.S. Cl. CPC G16H 50/20; G16H 50/70; G16H 40/20

(57) ABSTRACT

A DRGs automatic grouping method based on convolutional neural network, including: collecting case data and grouping according to a major diagnostic broad categories and core diagnosis-related grouping method; performing numerical coding to the data; constructing a shallow convolutional neural network model, using a k-means clustering method to cluster the feature vectors extracted from the convolutional network to obtain k category labels, combining the category labels and classifier to supervise the network performing iterative training; after finishing training the model, perform data grouping application. The method of the present disclosure is used to avoid the disadvantages of manual feature selection and additional data labeling for adding new grouping categories, automatic learning grouping can be performed for data with vague and difficult groupings.

### FIELD OF TECHNOLOGY

The present disclosure belongs to computer medical technology field, and especially relates to a DRGs (Diagnosis Related Groups) automatic grouping method based on a convolutional neural network.

### BACKGROUND

Due to the current aging population and the development of new science and technology, the deficiencies of the post-payment system of the health insurance fund tend to stimulate excessive medical services, and the prepayment system tends to prevaricate severe patients thereby reduce medical services, which have caused the total health costs to keep rising, the expenditures of the medical benefits fund to rise significantly, and medical benefits fund in many regions faces the risk of fund shortage.

DRGs (Diagnosis Related Groups) is a case combination method, and performs grouping on the cases mainly based on a principle of similar clinical courses and similar cost consumption. Making payments and performing targeted treatments according to diseases of different groupings to avoid waste of medical resources. However, due to the uneven economic development and medical care level, the population structure, health status and economic development level vary in different regions, so it is necessary to establish a grouping system adapted to local characteristics, and adjust the grouping system according to the operation results.

Chinese patent document with publication number CN110289088A discloses a method and a system for big data intelligent management based on DRGs, including: putting the inpatient case home page data of the yearly inpatient cases of a hospital in a region into a DRG grouper, grouping them according to DRG grouping principles (according to disease diagnosis, surgical operation, complications/complications, age, severity, etc.) to obtain n DRG groups and the distribution of the number of weights and cases, corresponding hospital days and costs for each DRG group; calculating the total number of weights of inpatient cases in the hospital; calculating the case mix index (CMI) value=total number of weights in the hospital/total number of inpatient cases in the hospital; calculating the relative weight RWi of the ith DRG group, and analyzing the proportion of cases with relative weight RWi>2 in the hospital to all cases in the hospital, the average cost of cases in DRG group i represents the average cost of the ith DRG group.

Chinese patent document with publication number CN107463771A discloses a method and a system for grouping cases, including: obtaining case information, grouping them into corresponding basic groups according to a major diagnosis codes and operation codes in the case information, and obtaining basic group codes and basic group names; when the major diagnosis corresponding to the major diagnosis codes does not belong to the inpatient time impact type, or, the basic group does not belong to a specific basic group, the diagnostic complexity score corresponding to each diagnosis code is then calculated based on the basic group code and each diagnosis code; the disease complexity index corresponding to the case information is calculated based on the diagnostic complexity score corresponding to each diagnosis code; the case information is divided into subgroups from the basic group based on the disease complexity index to obtain the diagnosis related groups code, diagnosis related groups name and diagnosis related groups relative weight to complete the case grouping.

However, the grouping of certain disease categories in each region may be controversial, different groupings may exist by using conventional methods, therefore there is an urgent need to design a method that can synthesize various actual information to divide categories that are relatively difficult to group.

### SUMMARY OF THE INVENTION

To solve the above-mentioned problems exist in the prior art, the present disclosure provides a DRGs automatic grouping method based on a convolutional neural network, which can synthesize actual information of the data for automatic division of disease types.

A DRGs automatic grouping method based on a convolutional neural network, comprising the following steps:

(1) collecting case data and dividing cases according to major diagnostic broad categories and a core disease diagnosis related grouping method, and dividing the case data into their corresponding groups as a training data set;

(2) performing numerical coding process to the case data in the training data set, and converting textual data into a corresponding numerical form;

(3) constructing a convolutional neural network model and performing iterative training on the model by using the data obtained from the step (2), during the training process, using a k-means clustering method to cluster feature vectors extracted from the convolutional neural network to obtain k category labels, combining the category labels and a classifier to supervise the convolutional neural network for iterative training; and

(4) after finishing training the model, performing numerical coding on the data to be divided and then inputting the data into the trained model for grouping.

The method of the present disclosure is used to avoid the disadvantages of manual feature selection and additional data labeling for adding new grouping categories, automatic learning grouping can be performed for data with vague and difficult groupings.

Wherein in the step (2), when performing the numerical coding, quantitating the case data and uniformly converting the case data to be within a range of 0 to 1 with the following conversion formula:

$$s = \frac{{V}_{c} - {V}_{min}}{{V}_{max} - {V}_{min}}$$

Wherein V_{c} is a current value to be calculated, Vₘᵢₙ, Vₘₐₓ, are the minimum and maximum values in the serial number, respectively.

Due to the relatively small amount of information in data relative to image, most of the popular network structures with relatively deep layers tend to cause data overfitting situation, wherein in the step (3), the shallow convolutional neural network with 3 convolutional layers is used for feature extraction on the data.

Wherein in the step (3), the process of training the convolutional neural network model is as follows:

(3-1), performing feature extraction on the coded data by using the convolutional neural network.

The convolutional calculation formula used for extracting features is as follows:

f(x,y) * g(x,y)=Σᵢ₌₋ₘ^{m}Σⱼ₌ₙⁿg(i,j)·f(x−i,y−j)

wherein f(x,y) is input data, g(x,y) is a convolution kernel function, and m and n are the convolution kernel length and width, respectively. The purpose of feature extraction is to synthesize different information of the data, and find the correlation between various information.

(3-2), introducing the feature vectors extracted by the convolutional neural network in the step (3-1) into a k-means clusterer to perform classification, calculating a distance between two categories of vectors by using cosine distance, dividing the closer ones into a class cluster, measuring a distance between class clusters by a distance between the shortest two points between all members of a class cluster to all members of another class cluster, finally taking a maximum distance between the class clusters as optimum efficiency, and selecting a corresponding k value automatically according to clustering efficiency;

The cosine distance is calculated by a formula as follows:

$$cos θ = \frac{a \cdot b}{‖ a ‖ \times ‖ b ‖}$$

wherein a and b are two different feature vectors.

(3-3), using the k categories obtained in the step (3-2) as labels for the data, using a regression model and a loss metric function to measure learning efficiency of the network, so as to supervise neural network learning until the network model converges.

Since there may be multiple categories of division, the regression model is a softmax method that can be used for multi-category problems, and is calculated by an approach as follows:

$${P ( z )}_{j} = \frac{{e}^{{Z}_{j}}}{{\sum }_{n = 1}^{N} {e}^{{Z}_{n}}} , j = 1 , 2 , … , N$$

wherein Zj is output of a jth neuron, N is a total quantity of categories, P(z)ⱼ is a probability value of the jth category; the model outputs a probability value for each category, thereby outputting N probability values for N categories.

The above-mentioned loss metric function is a cross entropy, and is calculated by a formula as follows:

L=Σᵢ₌₁^{M}yⁱ×log^{ŷ}^{ⁱ} +(1−yⁱ)×log^{1-ŷ}^{ⁱ}⁾

wherein yⁱ is the label of a ith category, ŷⁱ is a probability value of being predicted as the ith category, M is the quantity of samples.

Compared with the prior art, the present disclosure has the following beneficial effects:

The method of present disclosure, combining a convolutional neural network with a k-means clustering method, taking advantages of convolutional neural network automatic feature extraction and automatic optimization to extract connection between various features, using the labels generated by the clustering method to act into the neural network classifier, and then supervises the training and learning of the neural network to form a method that can achieve automatic optimizing grouping efficiency. For situations where it is difficult to group using conventional grouping rules, the method can combine all the information of the actual data for grouping, and can add data to optimize the grouping efficiency without additional workload.

### BRIEF DESCRIPTION OF THE DRAWINGS

FIG. 1 is a flow schematic diagram of the present disclosure of a DRGs automatic grouping method based on a convolutional neural network.

### DESCRIPTION OF THE EMBODIMENTS

The present disclosure is described in further detail below with a drawing and an embodiment, it should be pointed that the embodiment mentioned below is intended to make the present disclosure more understandable and do not limit it in any way.

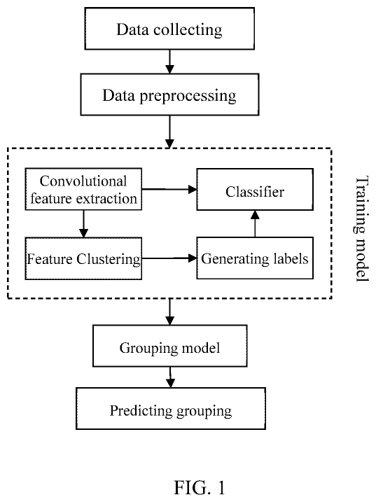

As shown in FIG. 1, a DRGs automatic grouping method based on a convolutional neural network, comprising the following steps:

S 1, collecting case data and dividing the cases according to major diagnostic broad categories and core diagnosis-related grouping methods into their corresponding groups. In this embodiment, the training data is performed in any of the core diagnosis related groups.

S2, performing coding on the data. The actual data is structured data is textually described, which needs to be coded into numerical form and input into a convolutional network for learning, quantitating the data and uniformly limiting the data within a range of 0 to 1.

S2-1, this implementation uses a 0, 1 approach to perform coding on the presence or absence of disease;

S2-2, for data with existing criteria such as department of consultation, blood type, surgery level and operation name, labeling each category using the ordinal numbers 0, 1, . . . , n by sorting the various categories, and then converting the ordinal number values to the values corresponding to 0 to 1, and is calculated by a formula as follows:

$$s = \frac{{V}_{c} - {V}_{min}}{{V}_{max} - {V}_{min}}$$

wherein V_{c} is a current value to be calculated, Vₘᵢₙ, Vₘₐₓ are the minimum and maximum values in the serial number, respectively.

Taking blood type as an example, the blood type column generally has 6 types of status that are A, B, O, AB, unknown and unchecked, which can be assigned serial numbers of 1, 2, 3, 4, 5 and 0 respectively, with A corresponding to serial number 0 and B corresponding to serial number 1, and the converted values are 0.2 and 0.4 respectively.

S2-3, for data in categories of age and treatment, the formula in the S2-2 is also applicable, the difference is that the minimum and maximum values are extracted from the data set to be trained.

S3, a convolutional neural network is constructed to iteratively train the data obtained from the S2, and k-means clustering is performed on the feature information output from the network to obtain k category labels, which is then combined with the classifier and the category labels of the network to supervise the neural network training.

S3-1, due to the relatively small amount of information in data relative to image, most of the popular network structures with relatively deep layers tend to cause data overfitting situation, therefore the embodiment choosing a network structure of the first 3 layers of the residual block of ResNet, convolution using a network composed of 1-dimensional convolutional kernels for performing feature extraction on the data, the convolutional approach can combine the information of various types of data with better semantic information, and is calculated by a formula as follows:

f(x,y) * g(x,y)=Σᵢ₌₋ₘ^{m}Σⱼ₌₋ₙⁿg(i,j)·f(x−i,y−j)

wherein f(x,y) is input data, g(x,y) is a convolution kernel function, and m and n are the convolution kernel length and width, respectively. The purpose of feature extraction is to synthesize different information of the data, and find the correlation between various information.

S3-2, the various feature information vectors output from the S3-1 are introduced into the k-means clustering method, and the distance between the various vectors is measured by using the cosine similarity method, and the clustering algorithm is optimized to classify the feature vectors into k categories.

Wherein the initial value of k for k-means is determined according to the grouping rules of the core disease diagnosis-related groups, for example, according to the grouping rules, the prior grouping disease and related operation grouping are initially divided into 9 groups, and when performing training on data of the group, initial value of k is set to be 9 tentatively. During the calculation of the clustering method, the k value is then adjusted by the clustering efficiency.

During the clustering training, using the principle of the distance between feature vectors to determine whether the feature vectors are the same class cluster, and if the distance between two feature vectors is small then they are the same class cluster, otherwise they are different class clusters. Measuring a distance between class clusters by a distance between the shortest two points between all members of a class cluster to all members of another class cluster, finally taking a maximum distance between the class clusters as optimum efficiency, and the cosine distance is used in the calculation to measure the distance between feature vectors, and is calculated by a formula as follows:

$$cos θ = \frac{a \cdot b}{‖ a ‖ \times ‖ b ‖}$$

wherein a and b are two different feature vectors.

S3-3, using the k categories obtained in the S3-2 as labels for the data, using a regression model and a loss metric function to measure learning efficiency of the network.

Since there may be multiple categories of division, the present disclosure is a softmax method that can be used for multi-category problems, and is calculated by a formula as follows:

$${P ( z )}_{j} = \frac{{e}^{{Z}_{j}}}{{\sum }_{n = 1}^{N} {e}^{{Z}_{n}}} , j = 1 , 2 , … , N$$

wherein Zj is output of a jth neuron, N is a total quantity of categories, P(z)ⱼ is a probability value of the jth category. The model outputs a probability value for each category, thereby outputting N probability values for N categories.

The above-mentioned loss metric function uses cross entropy, and is calculated by a formula as follows:

L=Σᵢ₌₁^{M}yⁱ×log^{ŷ}^{ⁱ}+(1−yⁱ)×log^{(1-ŷ}^{ⁱ}⁾

wherein yⁱ is the label of an ith category, ŷⁱ is a probability value of being predicted as the ith category, M is the quantity of samples. Iteratively training the network by the direction of minimizing the loss metric function, so as to achieve the best classification efficiency for the network.

In the specific application, performing numerical coding on the data to be divided and then inputting the data into the classification model, which automatically divides corresponding groupings.

The above-mentioned embodiment provides a detailed description of the technical solutions and beneficial efficiencies of the present disclosure, it should be understood that the above-mentioned is only specific embodiments of the present disclosure and is not intended to limit the present disclosure, and any modifications, additions and equivalent replacements made within the scope of the principles of the present disclosure shall be included in the scope of protection of the present disclosure.

What is claimed is:

1. A DRGs (Diagnosis Related Groups) automatic grouping method based on a convolutional neural network, the method comprising:
(1) collecting case data and dividing cases according to major diagnostic broad categories and a core disease diagnosis related grouping method, and dividing the case data into their corresponding groups as a training data set;
(2) performing numerical coding process to the case data in the training data set, and converting textual data into a corresponding numerical form;
(3) constructing a convolutional neural network model and performing iterative training on the model by using the data obtained from the step (2), during the training process, using a k-means clustering method to cluster feature vectors extracted from the convolutional neural network to obtain k category labels, combining the category labels and a classifier to supervise the convolutional neural network for iterative training; and
(4) after finishing training the model, performing numerical coding on the data to be divided and then inputting the data into the trained model for grouping.

2. The DRGs automatic grouping method based on a convolutional neural network according to claim 1, wherein in the step (2), when performing the numerical coding, quantitating the case data and uniformly converting the case data to be within a range of 0 to 1 with the following conversion formula:
$$s = \frac{{V}_{c} - {V}_{min}}{{V}_{max} - {V}_{min}}$$
wherein V_{c}, is a current value to be calculated, Vₘᵢₙ, Vₘᵢₙ, are the minimum and maximum values in the serial number, respectively.

3. The DRGs automatic grouping method based on a convolutional neural network according to claim 1, wherein in the step (3), using a shallow convolutional neural network with 3 convolutional layers to perform feature extraction on the data.

4. The DRGs automatic grouping method based on a convolutional neural network according to claim 1, wherein in the step (3), the process of training the convolutional neural network model is as follows:
(3-1), performing feature extraction on the coded data by using the convolutional neural network;
(3-2), introducing the feature vectors extracted by the convolutional neural network in the step (3-1) into a k-means clusterer to perform classification, calculating a distance between two categories of vectors by using cosine distance, dividing the closer ones into a class cluster, measuring a distance between class clusters by a distance between the shortest two points between all members of a class cluster to all members of another class cluster, finally taking a maximum distance between the class clusters as optimum efficiency, and selecting a corresponding k value automatically according to clustering efficiency;
(3-3), using the k categories obtained in the step (3-2) as labels for the data, using a regression model and a loss metric function to measure learning efficiency of the network, so as to supervise neural network learning until the network model converges.

5. The DRGs automatic grouping method based on a convolutional neural network according to claim 4, wherein in the step (3-1), a convolutional calculation formula used for extracting features is as follows:
$$f ( x , y ) * g ( x , y ) = {\sum }_{i = - m}^{m} {\sum }_{j = - n}^{n} g ( i , j ) \cdot f ( x - i , y - j )$$
wherein f(x,y) is input data, g(x,y) is a convolution kernel function, and m and n are the convolution kernel length and width, respectively.

6. The DRGs automatic grouping method based on a convolutional neural network according to claim 4, wherein in the step (3-2), the cosine distance is calculated by a formula as follows:
$$cos θ = \frac{a \cdot b}{‖ a ‖ \times ‖ b ‖}$$
wherein a and b are two different feature vectors.

7. The DRGs automatic grouping method based on a convolutional neural network according to claim 4, wherein in the step (3-3), the regression model is a softmax method, and is calculated by a formula as follows:
$${P ( z )}_{j} = \frac{{e}^{{Z}_{j}}}{{\sum }_{n = 1}^{N} {e}^{{Z}_{n}}} , j = 1 , 2 , … , N$$
wherein Zⱼ is output of a jth neuron, N is a total quantity of categories, P(z)ⱼ is a probability value of the jth category; the model outputs a probability value for each category, thereby outputting N probability values for N categories.

8. The DRGs automatic grouping method based on a convolutional neural network according to claim 4, wherein in the step (3-3), the loss metric function is a cross entropy, and is calculated by a formula as follows:
$$L = {\sum }_{i = 1}^{M} {y}^{i} \times {log}^{{\hat{y}}^{i}} + ( 1 - {y}^{i} ) \times {log}^{( 1 - {\hat{y}}^{i} )}$$
wherein yⁱ is the label of a ith category, ŷⁱ is a probability value of being predicted as the ith category, M is the quantity of samples.
